(19) United States
(12) Patent Application Publication
(10) Pub. No.: US 2004/0133221 A1
(43) Pub. Date: Jul. 8, 2004

(54) Connector assembly for joining a graft vessel to a side of a target vessel

(76) Inventors: Gregory E. Sancoff, North Hampton, NH (US); Jack Robertson, Cheshire, CT (US); Frederic P. Field, North Hampton, NH (US)

Correspondence Address: Pandiscio & Pandiscio, 470 Totten Pond Road, Waltham, MA, 02451-1914, US

(21) Appl. No.: 10/667,845

(22) Filed: Sep. 22, 2003

Related U.S. Application Data
(60) Provisional application No. 60/412,592, filed on Sep. 20, 2002.
(60) Provisional application No. 60/455,363, filed on Mar. 17, 2003.

(51) Int. Cl. A61B 17/08

(52) U.S. Cl. 606/153

(57) ABSTRACT

A connector assembly for joining a graft vessel to a target vessel. The assembly includes a tubular connector member provided with tines at a distal end thereof, the tines extending inwardly of the connector member and then radially outwardly and then proximally at sharp ends thereof, the tines being adapted to engage end portions of the graft vessel and thereafter wall portions of the target vessel, a spreader portion comprising a tubular body slidably disposed in the tubular connector member and movable to engage the inwardly extending tines and force the tines into position substantially disposed in a hypothetical extension of walls of the connector member, and a seal portion slidably disposed around the connector member and provided with flanges for engagement with the target vessel in wall areas proximate the tines to clamp target vessel wall portions to the tines.

### CROSS-REFERENCE TO RELATED APPLICATIONS

This patent application claims benefit of:

(1) pending prior U.S. Provisional Patent Application Serial No. 60/412,592, filed Sep. 20, 2002 by Gregory E. Sancoff et al. for A CONNECTOR ASSEMBLY FOR JOINING A GRAFT VESSEL TO A SIDE OF A TARGET VESSEL (Attorney's Docket No. ONUX-35 PROV); and

(2) U.S. Provisional Patent Application Serial No. 60/455,363, filed Mar. 17, 2003 by Gregory E. Sancoff et al. for A CONNECTOR ASSEMBLY FOR JOINING A GRAFT VESSEL TO A SIDE OF A TARGET VESSEL (Attorney's Docket No. ONUX-35 A PROV).

The two above-identified patent applications are hereby incorporated herein by reference.

### FIELD OF THE INVENTION

This invention generally relates to apparatus and methods for surgery. More specifically, this invention relates to apparatus and methods for the surgical anastomosis of physiological vessels, and particularly for joining a graft vessel to a target vessel.

### BACKGROUND OF THE INVENTION

It is a common surgical procedure to join together two or more surgical vessels, such as intestines or blood vessels. The three main types of connections include: end-to-end, end-to-side, and side-to-side connections. As these structures often carry fluid, the connections formed must generally be at least substantially complete around the entire surface of the joinder. Traditional means for connecting together these structures include the use of sutures or staples.

Placing sutures by hand around the circumference of a vessel is often very difficult and cumbersome due to various factors. These factors include space limitations at the typical surgical site (e.g., at an interior surgical site); limited angles of approach to the surgical site; and the nature of the attachment of the desired surgical vessel to, or containment within, various other structures. Such limitations typically cause difficulty in accessing remote sides of the desired anatomical vessels and impede the manipulation of surgical instruments at the surgical site.

Additionally, blood vessels such as the coronary arteries, or those vessels used to form bypasses, are typically fairly small in diameter and have very thin walls. The thin walls frequently cause these vessels to adopt a collapsed configuration during handling, which in turn causes difficulty in handling and positioning the vessels.

Accordingly, there is a need for an improved apparatus and method for joining a graft vessel to an opening in a target vessel.

### SUMMARY OF THE INVENTION

An object of the present invention is, therefore, to provide an apparatus for joining a graft vessel to a target vessel and to provide a method for joining a graft vessel to a target vessel.

With the above and other objects in view, a feature of the present invention is the provision of a connector assembly for joining a graft vessel to an opening in a target vessel. The connector assembly comprises a tubular connector member provided with a plurality of tines at a distal end thereof, the tines in an unstressed state extending inwardly toward a central axis of the tubular connector member and then substantially radially outwardly from the central axis of the tubular connector member and then proximally at sharp ends thereof, the tines being adapted to engage end portions of the graft vessel and thereafter wall portions of the target vessel; and a spreader portion comprising a tubular body slidably disposed in the tubular connector member and movable in the tubular connector member so as to engage the inwardly extending tines and force the tines of the tubular connector member into positions substantially disposed in a hypothetical extension of walls of the tubular connector member. A seal portion is slidably disposed around the tubular connector member and is provided with flanges at a distal end thereof for engagement with the target vessel in wall areas proximate the tines to clamp the target vessel wall portions to the tines.

In accordance with a further feature of the invention, there is provided a method for joining a graft vessel to a target vessel. The method includes the steps of providing a connector assembly having graft supporting structure at a distal end thereof for supporting an everted end of the graft vessel and pressure exerting structure at another portion thereof for exerting distally directed pressure on the graft vessel everted end, providing a deployer assembly for holding, manipulating, and releasing the connector assembly, attaching the connector assembly to the deployer assembly, extending the graft vessel through the deployer assembly and the connector assembly, with a distal end of the graft vessel extending distally of the distal end of the connector assembly, everting the distal end of the graft vessel back upon the connector assembly graft supporting structure, producing an opening in the target vessel if one is not already available, manipulating the deployer assembly to move the everted end of the graft vessel through the target vessel opening, into the target vessel, and into engagement with an interior wall of the target vessel around the opening therein, manipulating the deployer assembly to move the connector assembly pressure exerting structure into engagement with an exterior wall of the target vessel proximate the everted end of the graft vessel, and disconnecting the deployer assembly from the connector assembly, whereby to hold the everted end of the graft vessel and an area of the target vessel adjacent the graft vessel between the connector assembly pressure exerting structure and the connector assembly graft supporting structure.

The above and other features of the invention, including various novel details of construction and combinations of parts and method steps, will now be more particularly described with reference to the accompanying drawings. It will be understood that the particular device and method embodying the invention are shown by way of illustration only and not as limitations of the invention. The principles and features of this invention may be employed in various and numerous embodiments without departing from the scope of the invention.

### BRIEF DESCRIPTION OF THE DRAWINGS

Reference is made to the accompanying drawings in which are shown illustrative embodiments of the invention, from which its novel features and advantages will be apparent.

In the drawings:

FIG. 1 is an exploded perspective view of one form of connector assembly illustrative of an embodiment of the invention;

FIG. 2 is a perspective view of the connector assembly of FIG. 1, showing the components of the assembly telescopically joined together;

FIG. 3 is a view similar to that of FIG. 2, but shows the connector assembly components further joined telescopically;

FIG. 4 is a perspective view of a deployer assembly and the connector assembly, shown separately;

FIG. 5 is a perspective view of the deployer assembly of FIG. 4, with the components thereof assembled;

FIGS. 6-8 illustrate mounting of the connector assembly on the deployer assembly;

FIGS. 9-14 diagrammatically illustrate steps in a method for joining a graft vessel to a target vessel, using the connector and deployer assemblies of FIGS. 1-5, and illustrative of an embodiment of the invention;

FIG. 15 is similar to FIG. 1, but illustrating an alternative embodiment of connector assembly components; and

FIGS. 16-18 diagrammatically illustrate steps in a method for joining a graft vessel to a target vessel, using an end-to-end fixation.

### DETAILED DESCRIPTION OF THE PREFERRED EMBODIMENTS

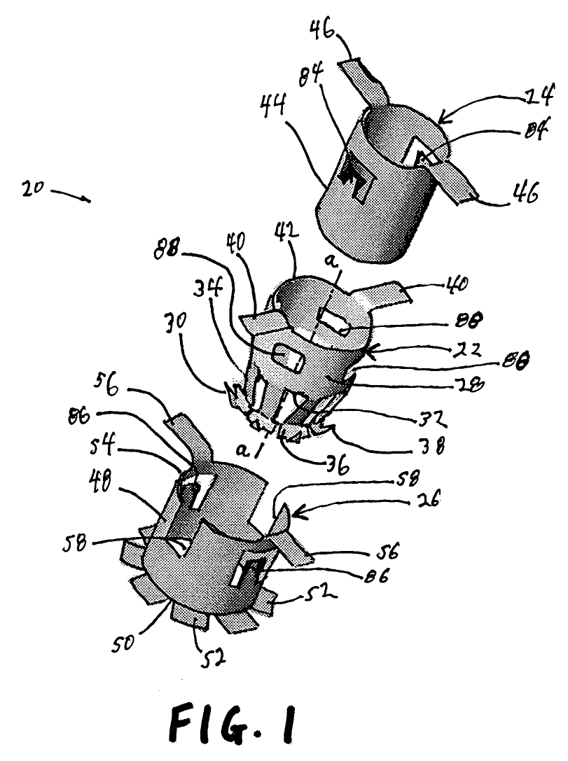

Referring to FIG. 1, it will be seen that a connector assembly 20 includes a tubular connector member 22, a spreader portion 24, and a seal portion 26.

The tubular connector member 22 is provided with a tubular body portion 28 and a plurality of tines 30 extending distally from a distal end 32 of the body portion 28. The tines 30 each include a leg portion 34 extending inwardly toward a central axis a-a of the tubular connector member 22, a flange portion 36 extending substantially radially outwardly from the central axis a-a of the tubular connector member 22, and a sharp end portion 38 extending proximally from an outer extremity of the flange portion 36. As will be further described herein below, the sharp end portions 38 of the tines are adapted to penetratingly engage an end portion of a graft vessel G (FIG. 10) and, thereafter, wall portions of a target vessel T (FIG. 13). The tubular connector member 22 is further provided with opposed radially outwardly extending flanges 40 (FIG. 1) at a proximal end 42 of the tubular connector member 22.

The spreader portion 24 includes a tubular body 44 which is adapted for sliding disposition in the tubular connector member 22. The spreader portion 24 is telescopically movable in the tubular connector member 22 so as to engage the inwardly extending tines 30 and force the tines 30 into positions substantially disposed in a hypothetical extension of the tubular body portion 28 of tubular connector member 22. The spreader portion 24 is provided with opposed outwardly extending flanges 46.

The seal portion 26 is adapted for sliding disposition around the tubular connector member 22 and the spreader portion 24. The seal portion 26 includes a tubular body 48 provided, at its distal end 50, with a ring of flanges 52 extending radially outwardly, and at its proximal end 54 with opposed outwardly extending flanges 56. The tubular body 48 of seal portion 26 is further provided with opposed slots 58 extending distally from the proximate end 54 of the tubular body 48.

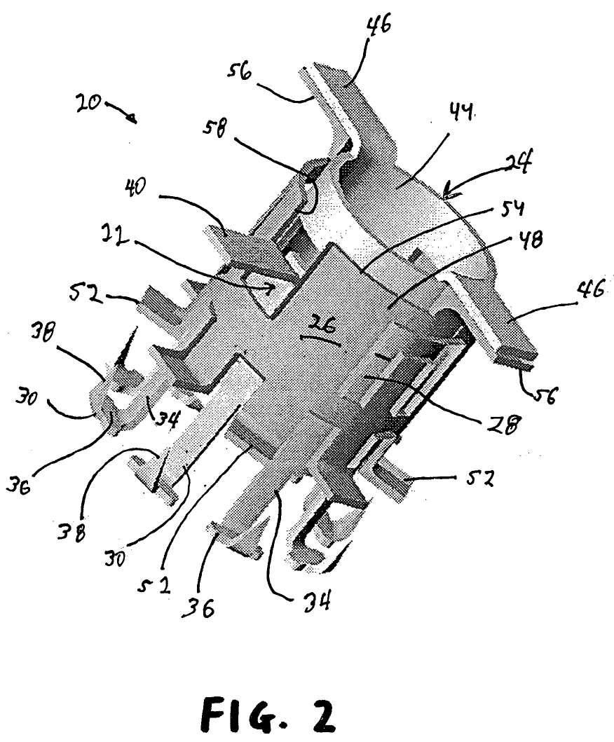

Referring now to FIG. 2, it will be seen that the spreader portion 24 is disposed within the tubular connector member 22. In FIG. 2, the spreader portion 24 is shown sufficiently advanced in the tubular connector member 22 to force the tines 30 into the aforesaid positions disposed in a hypothetical extension of the tubular body portion 28 of tubular connector member 22.

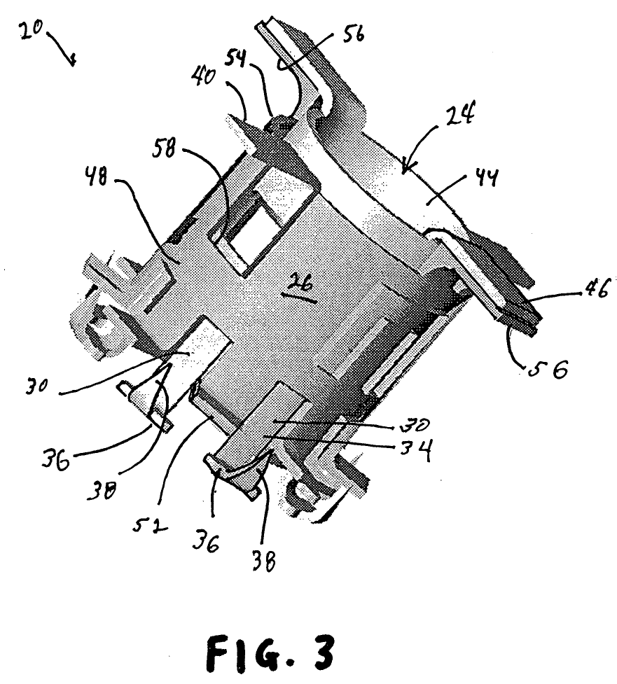

In FIG. 3 there is shown the same assembly as is shown in FIG. 2, but with the seal portion 26 advanced distally so as to position the flanges 52 of the seal portion 26 proximate the flange portions 36, and sharp end portions 38, of the tubular connector member 22.

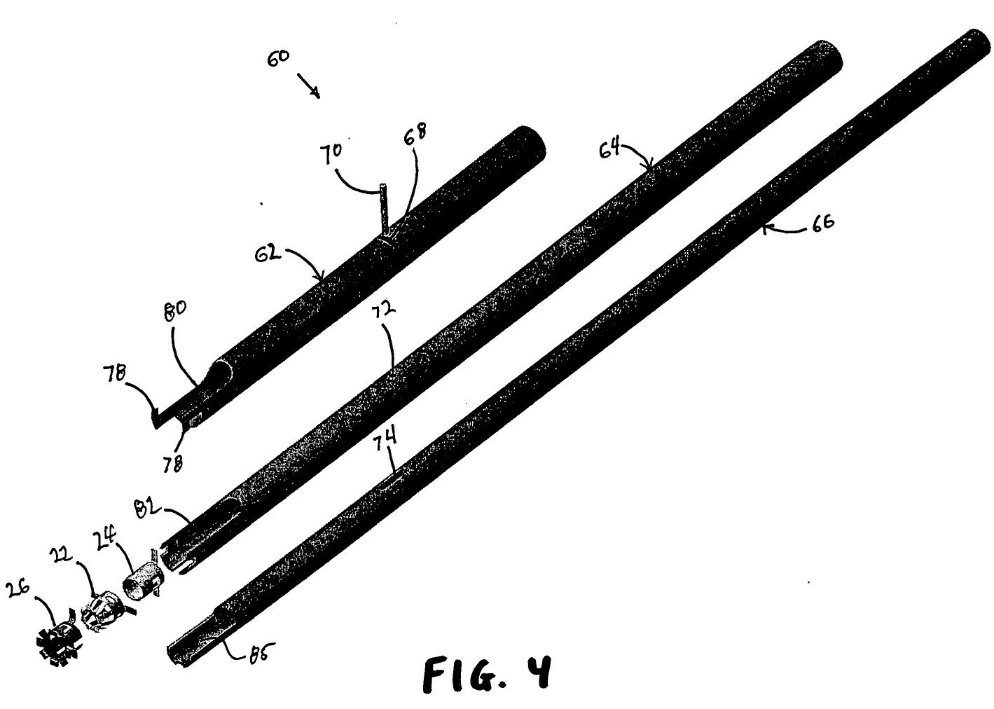

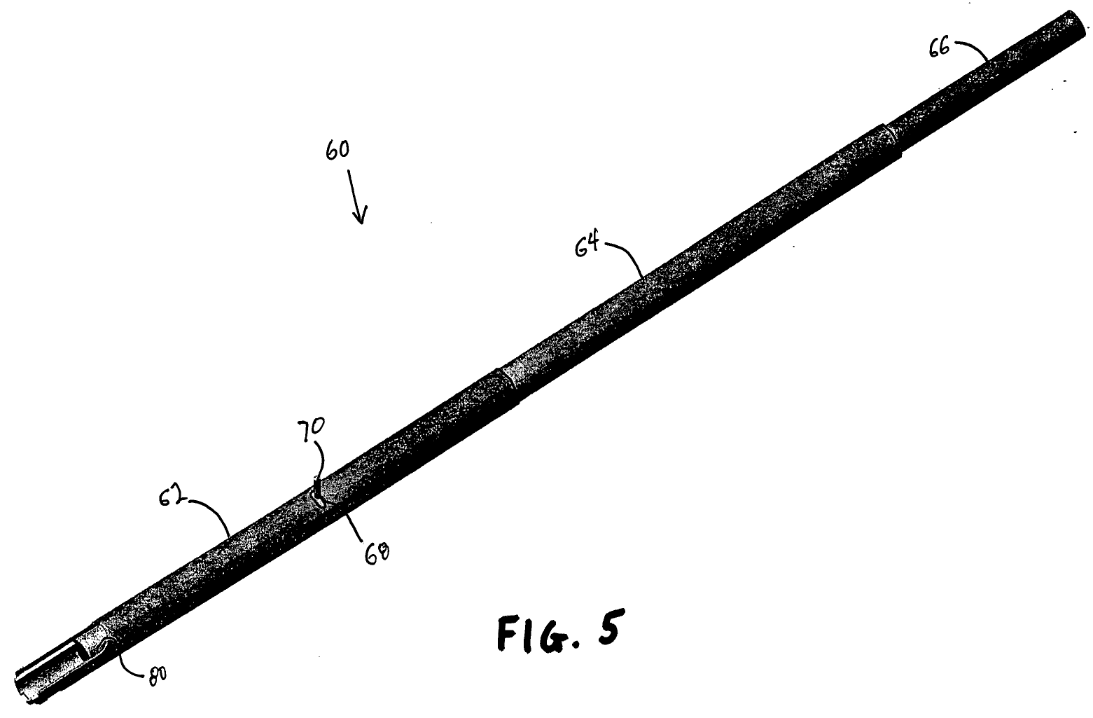

In FIG. 4 there are shown components of a deployer assembly 60, including a grabber 62, a holder 64, and a pusher 66. The grabber 62 is provided with a widthwise slot 68 for receiving a pin 70. The holder 64 is provided with an aperture 72 therethrough which is alignable with the grabber slot 68 and configured to receive the pin 70. The pusher 66 is provided with a lengthwise slot 74 for receiving the pin 70. Thus, the grabber 62, holder 64, and pusher 66 are connected together (FIG. 5) by the pin 70 which, in combination with the slots 68, 74, permits limited rotative movement of the grabber 62 and limited axial movement of the pusher 66.

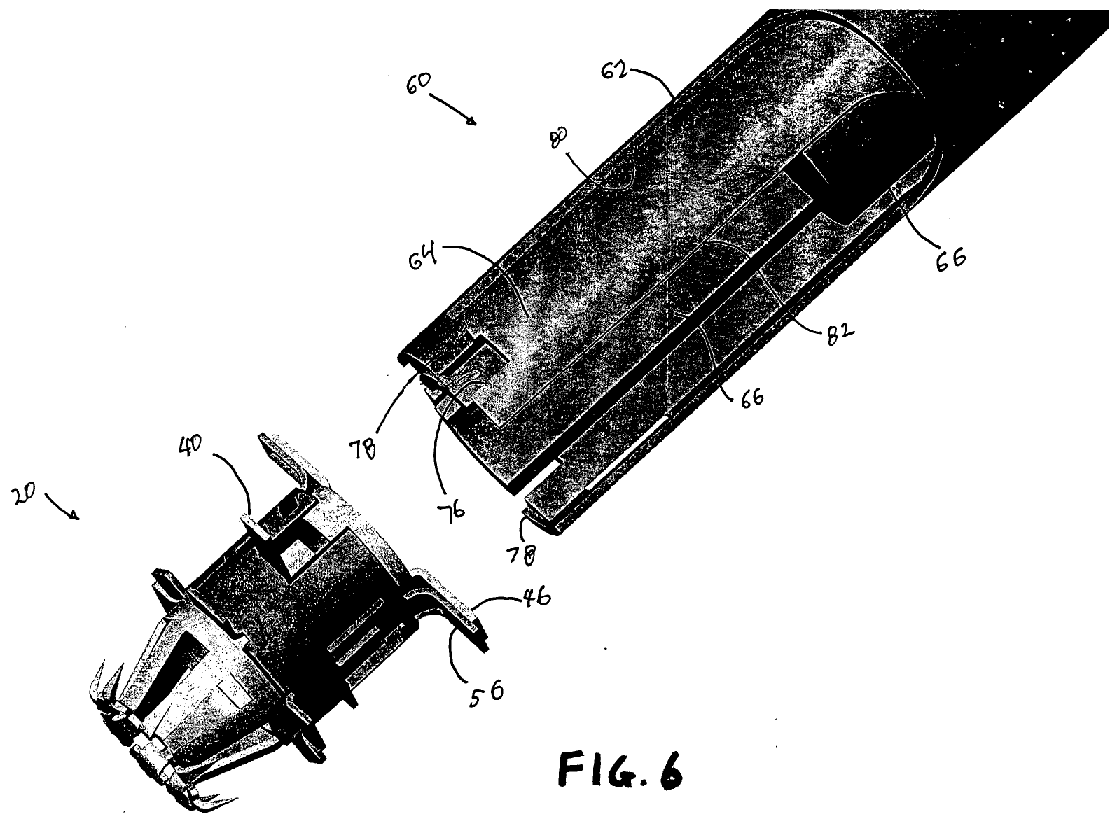

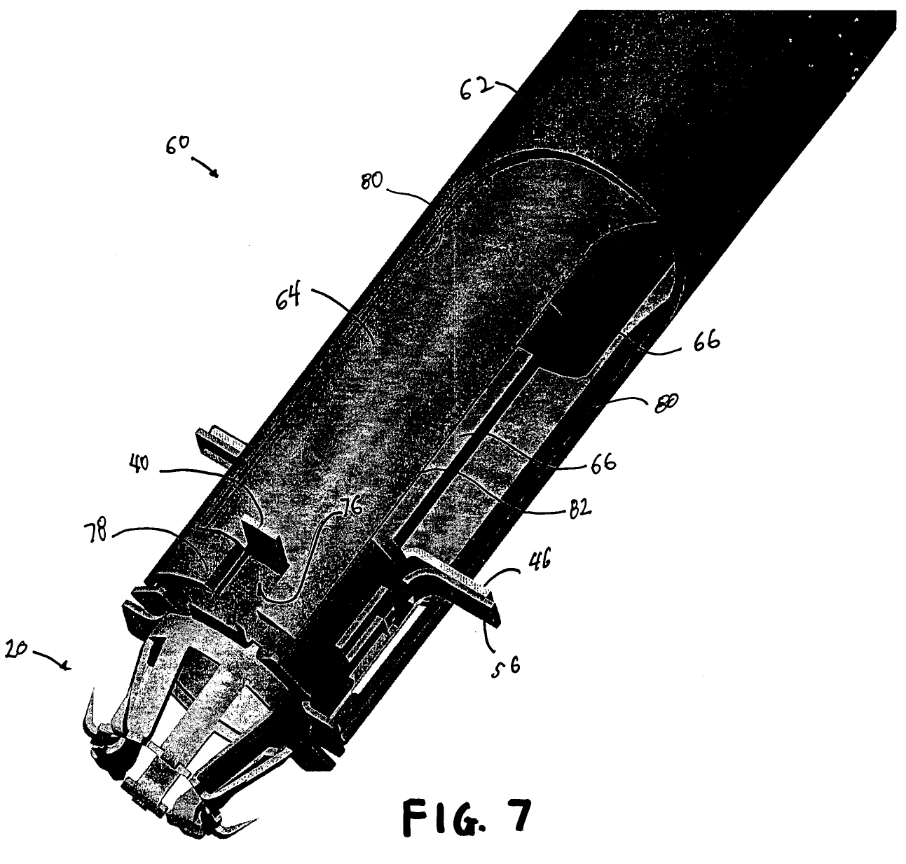

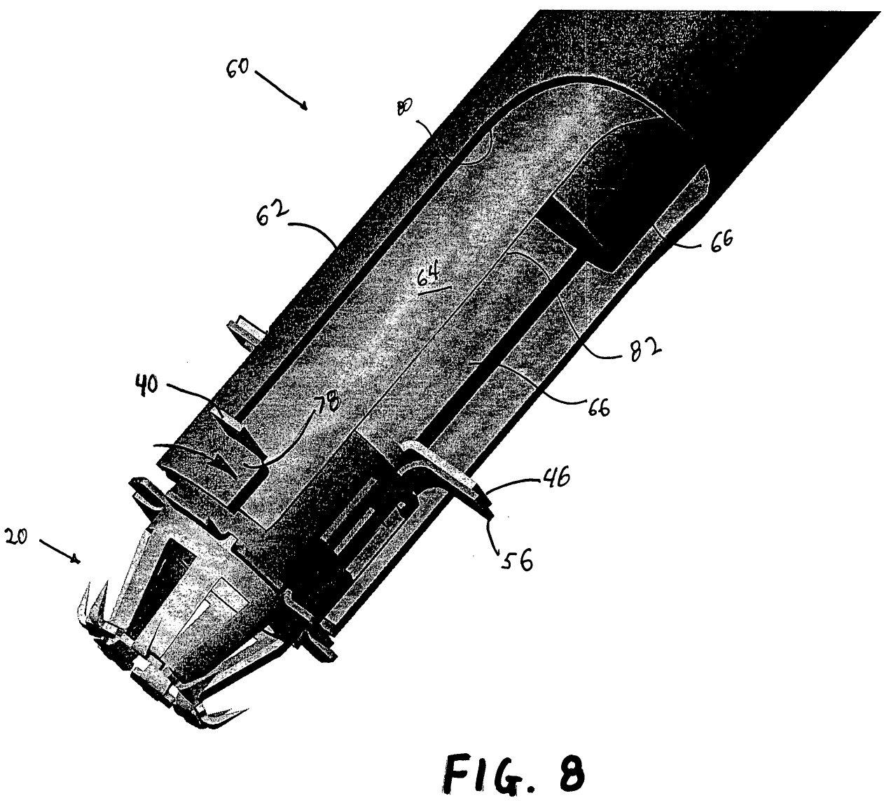

As shown in FIG. 6, the holder 64 is provided with slots 76 adapted to receive the flanges 40 of tubular connector member 22 when the connector assembly 20 is inserted in the distal end of the deployer assembly 60 (FIG. 7). The grabber 62 is provided with widthwise extending lugs 78 which, upon rotation of the grabber 62 (FIG. 8), slide under the flanges 40 of tubular connector member 22 so as to hold the tubular connector member 22 in the deployer assembly 60.

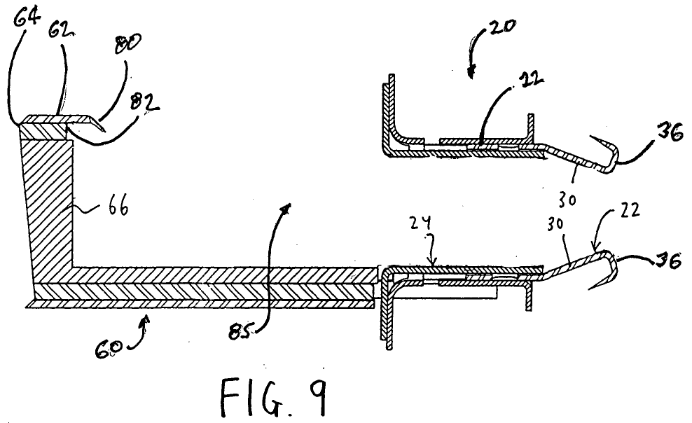

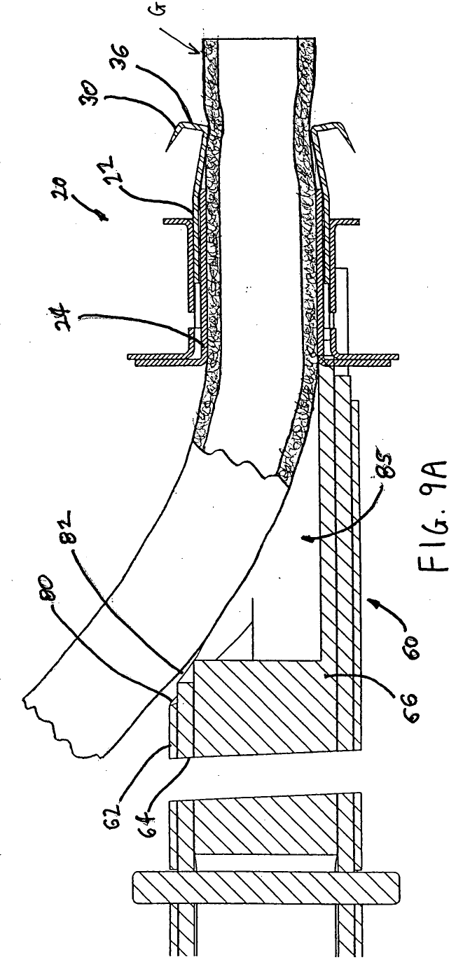

FIGS. 9-14 illustrate one preferred manner for using connector assembly 20 to join the end of a graft vessel G to the side of a target vessel T.

Referring next to FIG. 9, it will be seen that in a preferred use of the connector assembly 20 in conjunction with the deployer assembly 60, pusher 66 is advanced distally (FIG. 9A) which causes spreader portion 24 to advance distally, whereby to spread the tines 30 apart and thereby facilitate loading a graft vessel onto connector assembly 20. More particularly, with the tines 30 opened up, the graft vessel G (FIG. 9A) is fed into the deployer assembly 60 by way of openings 80, 82 in sides of the grabber 62 and holder 64, respectively. The graft vessel G is further extended through a concavity 85 in the pusher 66 and through the connector assembly 20 (with its opened-up tines 30) and slightly beyond. Once this has been done, pusher 66 is withdrawn proximally (FIG. 9B) so as to permit tines 30 to return inwardly again, whereby to facilitate eversion of the graft vessel G over the tine sharp end portions 38.

More particularly, as shown in FIG. 10, the graft vessel G is everted back over the tine sharp end portions 38 and perforated thereby. Significantly, eversion can be effected with reduced trauma to the graft vessel G since the retraction of pusher 66 permits tines 30 to return to their inboard position, thus causing less stress on the graft vessel during eversion. Also significantly, during eversion and thereafter, the flange portions 36 (FIG. 2) of tines 30 help support and carry the everted graft tissue, including preventing the vessel from sliding radially inwardly and/or upwardly along the tines, which could affect the integrity of the vessel joinder and/or expose the connector to blood flow.

A slot S (FIG. 11) is then cut in a wall of target vessel T and the tines 30 of the connector member 22 are inserted through the slot S and into the target vessel.

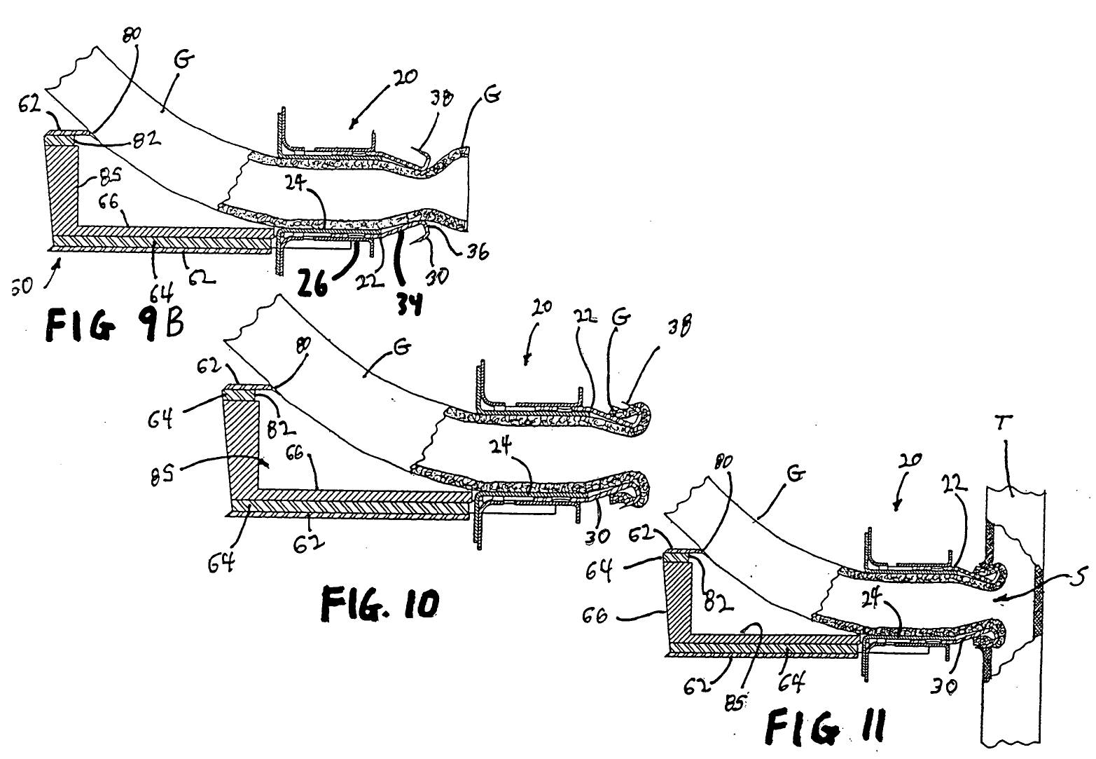

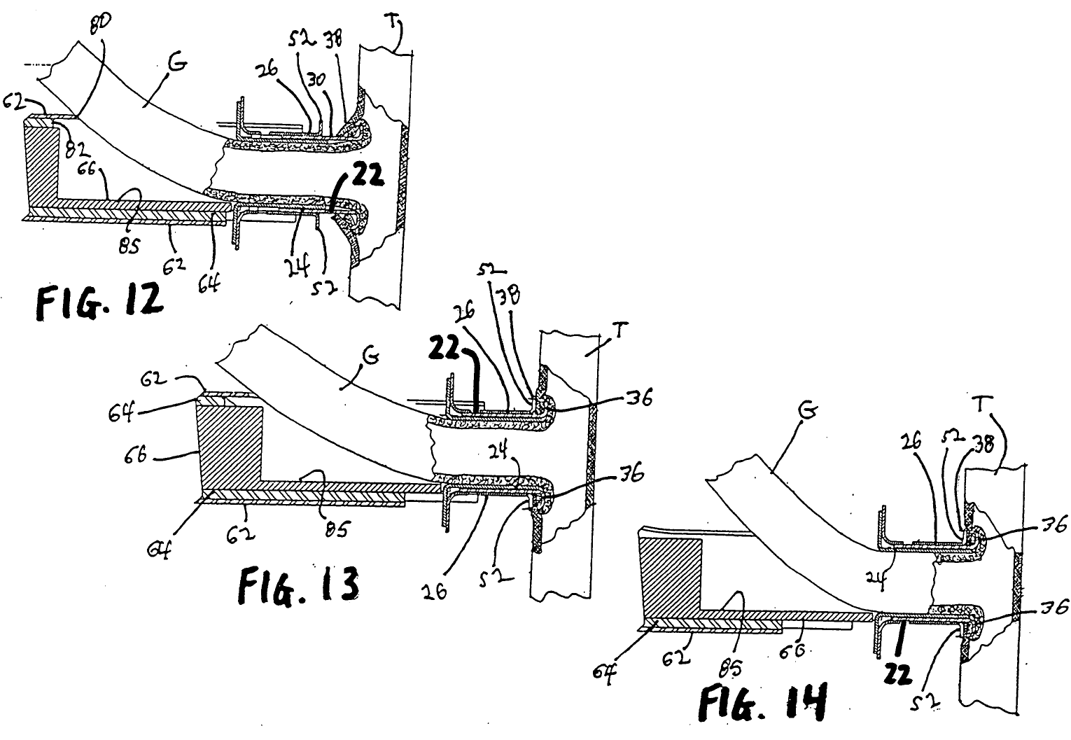

The pusher 66 is then pushed toward the target vessel T, which causes the spreader portion 24 to engage the connector member tines 30 to spread the tines radially outwardly (FIG. 12). The deployer assembly 60 is then pulled proximally slightly to cause the tine sharp end portions 38 to engage the target vessel T.

The pusher 66 is then again pushed distally (FIG. 13), causing distal movement of the seal portion 26 to bring the seal portion flanges 52 into engagement with the target vessel T proximate the tine flange portions 36 and sharp end portions 38 (FIGS. 13 and 14). At this point, tabs 84 (FIG. 1) on the spreader portion 24, and tabs 86 on the seal portion 26, snap into slots 88 in the connector member 22. The tabs 84 and 86 are bent slightly outwardly and inwardly, respectively, to engage the slots 88. The tabs 84, 86 can interface with a multitude of slots 88 to provide a selection of gaps between the tine flange portions 36 and the seal portion flanges 52, to accommodate different thicknesses of tissue.

Upon completion of the vessel joining operation, the grabber 62 is rotated to release the connector assembly 20 and the pusher 66 is pushed distally to eject the connector assembly 20 from the deployer assembly 60.

Significantly, due to the manner in which connector assembly 20 engages graft vessel G and target vessel T, there is substantially no contact between the connector assembly and blood flowing in graft vessel G and/or blood flowing in target vessel T, thus minimizing stenosis or other lumen occlusion, blood leakage, infection, adverse tissue reactions, blood flow turbulence, blood clotting and the like.

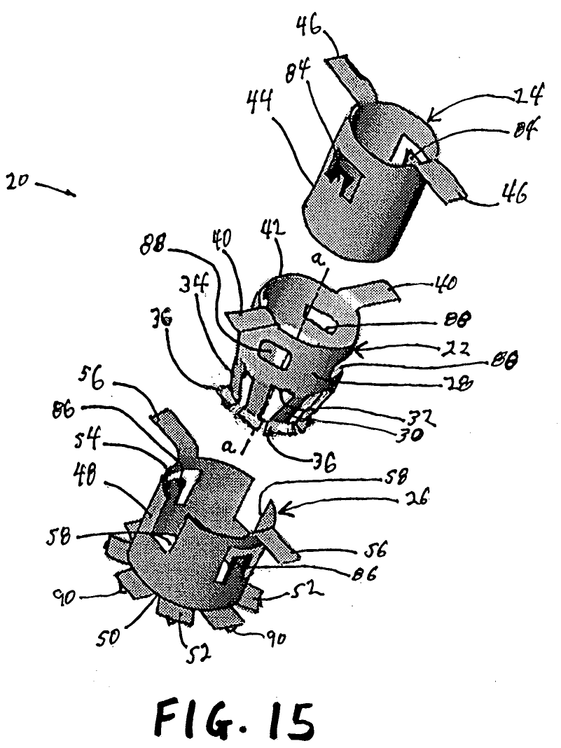

In some instances, as when working with particularly delicate vessels, the connector member 22 may be provided without the sharp end portions, as shown in FIG. 15. In this case, the connector assembly 20 effects the connection between the two vessels G, T by the tine flanges 36 and the seal portion flanges 52 pressing from opposite sides on the outer wall surface of the target vessel T and the everted portion of the graft vessel. Similarly, the seal portion 26 may be provided with distally-extending sharp end portions 90 in addition to, or in place of, the flanges 52.

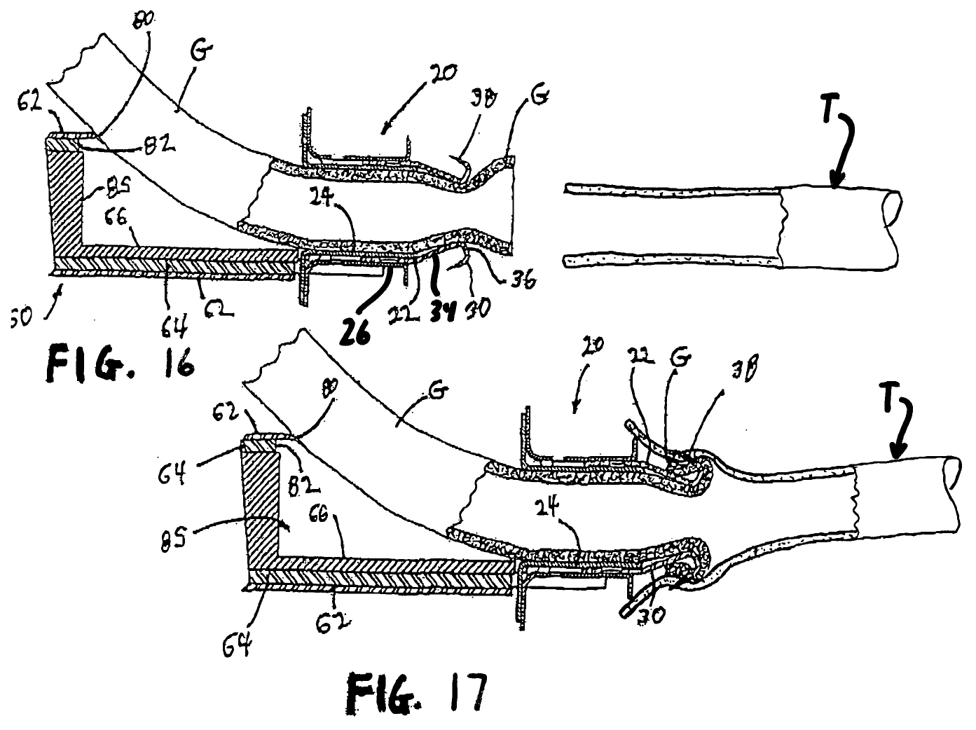

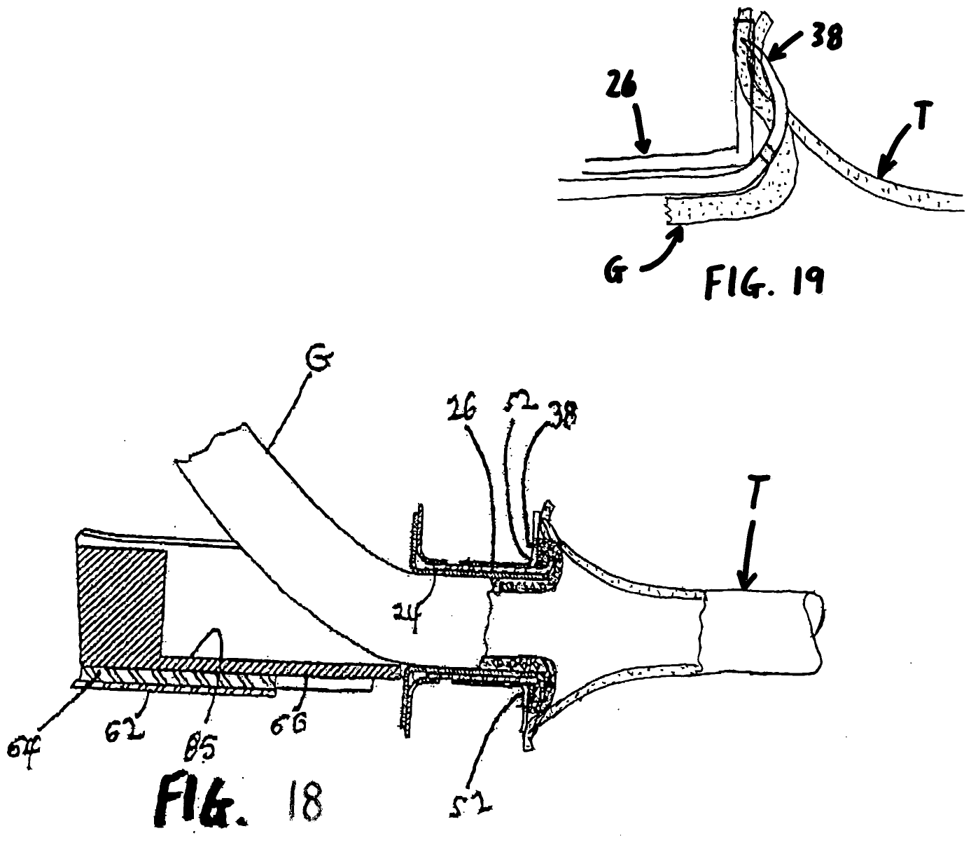

It is also possible to use connector assembly 20 to attach the end of a graft vessel to the end of a target vessel. See FIGS. 16-18. In this respect it should be appreciated that because the tines are between the sealing flanges, the flanges can press the two vessel layers down against the tines more securely. Connector assembly 20 can also be used for angled approaches as well as the end-to-side and end-to-end connections described above.

Connector assembly 20 can be formed out of any biocompatible materials having characteristics consistent with the present invention. By way of example but not limitation, connector assembly 20 can be formed out of metals (including shape memory alloys) plastics, bioabsorbable materials, etc.

Connector assembly 20 can be used to connect various types of vessels including, but not limited to, blood vessels, intestine, or other tubular structures.

There is thus provided an improved apparatus and method for connecting a graft vessel to a target vessel without the need for the usual suturing or stapling procedures.

It is to be understood that the present invention is by no means limited to the particular construction and method steps herein disclosed and/or shown in the drawings, but also comprises any modification or equivalent within the scope of the disclosure. For example, in manufacture of the connector assembly, the spreader portion 24 and the seal portion 26 may be formed as a single integral unit or connected together, as by spot welds 92 connecting the flanges 46,56 (FIG. 3).

What is claimed is:

1. A connector assembly for joining a graft vessel to a target vessel, the connector assembly comprising:
a tubular connector member provided with a plurality of tines at a distal end thereof, the tines in an unstressed state extending inwardly toward a central axis of the connector member, the tines being provided at distal ends thereof with vessel engaging structure;
a spreader portion comprising a tubular body slidably disposed in said connector member and movable in the connector member to engage the tines and extend the tines in directions outwardly from the connector member central axis; and
a seal portion slidably disposed around said connector member and provided with vessel engaging structure at a distal end thereof;
wherein the connector member vessel engaging structure is adapted to engage end portions of the graft vessel within the target vessel and urge the graft vessel end portions proximally, and the seal portion vessel engaging structure is adapted to engage wall portions of the target vessel adjacent the graft vessel and urge the target vessel wall portions distally toward the graft vessel end portions.

2. The connector assembly in accordance with claim 1 wherein each of the tines comprises a leg portion inclining inwardly toward the connector member central axis and the connector member vessel engaging structure comprises at least in part a flange portion at a distal end of each of said leg portions and extending in a direction substantially radially outwardly from the connector member central axis for engagement with the graft vessel end portions.

3. The connector assembly in accordance with claim 2 wherein the connector member vessel engaging structure further comprises a sharp end portion extending from a distal end of each of said tine flange portions.

4. The connector assembly in accordance with claim 3 wherein said sharp end portions extend proximally from said tine flange portion.

5. The connector assembly in accordance with claim 1 wherein said spreader portion is adapted to force the tines into positions substantially disposed in a hypothetical extension of tubular walls of said connector member.

6. The connector assembly in accordance with claim 1 wherein the seal portion vessel engaging structure comprises flanges at a distal end thereof for engagement with target vessel wall areas proximate the graft vessel.

7. The connector assembly in accordance with claim 6 wherein the seal portion vessel engaging structure further comprises sharp end portions protruding from said flanges.

8. The connector assembly in accordance with claim 1 wherein said seal portion vessel engaging structure comprises sharp end portions extending from distal ends of said seal portions.

9. The connector assembly in accordance with claim 1 wherein said spreader portion and said seal portion are connected together.

10. A connector assembly for joining a graft vessel to an opening in a target vessel, the connector assembly comprising:
a tubular connector member provided with a plurality of tines at a distal end thereof, the tines extending inwardly toward a central axis of the connector member and then substantially radially outwardly from the central axis of the connector member and then proximally at sharp ends thereof, the tines being adapted to engage end portions of the graft vessel and thereafter wall portions of the target vessel;
a spreader portion comprising a tubular body slidably disposed in the tubular connector member and movable in the tubular connector member to engage the inwardly extending tines and force the tines into positions substantially disposed in a hypothetical extension of walls of the tubular connector member; and
a seal portion slidably disposed around the tubular connector member and provided with flanges at a distal end thereof for engagement with the target vessel in wall areas proximate the tines to clamp target vessel wall portions to the tines.

11. A connector assembly according to claim 10, wherein the portions of the connector assembly shield the connector assembly from blood flow.

12. A deployer assembly for effecting operation of a connector assembly for joining a graft vessel to an opening in a target vessel, the connector assembly comprising:
a connector member provided at a distal end thereof with tines each provided at a distal end thereof with vessel engaging structure;
a spreader portion movable in the connector member to engage the tines and extend the tines outwardly from a connector member central axis; and
a seal portion movable on the connector member and provided with vessel engaging structure at a distal end thereof;
the deployer assembly comprising:
a grabber comprising an elongated tubular member having an open distal end and a side opening proximate the grabber distal end, the side opening being adapted to receive the graft vessel and the open distal end being adapted for passing an end of the graft vessel therethrough to extend distally therefrom, said grabber further comprising lugs for locking the connecting member in said grabber;
a holder disposed within said grabber and having a side opening adapted for alignment with the grabber side opening for receiving the graft vessel, said holder having an open distal end, and slots adapted to receive connector member flanges;
said grabber being rotatable on said holder to cause said grabber lugs to slide into position to lock in place the connector member flanges to hold the connector member in the deployer assembly; and
a pusher disposed in said holder and having a distal portion engagable with the connector assembly spreader portion to move the spreader portion into engagement with the tines and to move said seal portion into engagement with a juncture of the graft vessel and the target vessel.

13. The deployer assembly in accordance with claim 12 wherein said grabber is provided with a widthwise slot, said holder is provided with an aperture for alignment with said grabber slot, and said pusher is provided with a lengthwise slot for alignment with the aperture, and the deployer assembly further comprises a pin extending through the grabber slot, the holder aperture, and the pusher slot, whereby said grabber is movable rotatably on said holder, and said pusher is movable axially in said holder, the rotatable movement of said grabber being limited by said pin in the grabber slot, and the axial movement of said pusher being limited by said pin in the pusher slot.

14. A method for joining a graft vessel to an opening in a target vessel, the method comprising the steps of:
providing a connector assembly having structure at a distal end thereof for supporting an everted end of the graft vessel and for exerting proximally directed pressure on the graft vessel everted end;
providing a deployer assembly for holding, manipulating, and releasing the connector assembly;
attaching the connector assembly to the deployer assembly;
extending the graft vessel through the deployer assembly and the connector assembly with a distal end of the graft vessel extending distally of the distal end of the connector assembly;
everting the distal end of the graft vessel back upon the connector assembly graft vessel supporting structure;
producing an opening in the target vessel if one is not already available;
manipulating the deployer assembly to move the everted end of the graft vessel through the target vessel opening, into the target vessel, and into engagement with an interior wall of the target vessel around the opening therein;
manipulating the deployer assembly to move the connector assembly pressure exerting structure into engagement with an exterior wall of the target vessel proximate the everted end of the graft vessel; and
disconnecting the deployer assembly from the connector assembly;
whereby to hold the everted end of the graft vessel and an area of the target vessel adjacent the graft vessel between the connector assembly pressure exerting structure and the connector assembly graft supporting structure.

15. The method in accordance with claim 14 wherein the connector assembly comprises a connector member having a body portion and tines extending from a distal end of the body portion, and a spreader portion for engaging the tines to move distal ends of the tines in directions radially outwardly from a centerline of the connector member, the tines having the vessel supporting structure at their distal ends; and
the deployer assembly comprises a pusher for engagement with the connector member; and
the step of manipulating the deployer assembly to move the end of the graft vessel into engagement with the interior wall of the target vessel comprises:
manipulating the pusher to move the connector assembly spreader portion into engagement with the tines to cause the tine vessel supporting structure to move radially outwardly; and
moving the deployer assembly proximally to cause the tine vessel supporting structure to move the end of the graft vessel into engagement with the interior wall of the target vessel.

16. The method in accordance with claim 15 wherein the tine vessel supporting structure comprises sharp end portions; and
the steps of moving the tine vessel supporting structure radially outwardly comprises moving the sharp end portions into piercing engagement with the graft vessel everted end portion; and
the step of moving the deployer assembly proximally comprises moving the tine sharp end portions into piercing engagement with the target vessel wall adjacent the graft vessel.

17. A method according to claim 14, wherein the everted end of the graft vessel shields the connector assembly from blood flow.

18. A method for joining a graft vessel to an opening in a target vessel, the method comprising the steps of:
providing a connector assembly having at a distal end thereof tines having sharp end portions at distal ends thereof for penetrating the graft vessel, for supporting an everted end of the graft vessel, and for exerting proximally directed pressure on the graft vessel everted end, and having a seal portion for exerting distally directed pressure on the target vessel wall;
providing a deployer assembly for holding, manipulating, and releasing the connector assembly;
attaching the connector assembly to the deployer assembly;
extending the graft vessel through the deployer assembly and the connector assembly with a distal end of the graft vessel extending distally of the distal end of the connector assembly;
everting the distal end of the graft vessel back upon the connector assembly sharp end portions;
producing an opening in the target vessel if one is not already available;
manipulating the deployer assembly to move the connector assembly tines and the everted end of the graft vessel through the target vessel opening, into the target vessel, and into engagement with an interior wall of the target vessel around the opening therein;
manipulating the deployer assembly to move the connector assembly seal portion pressure exerting structure into engagement with an exterior wall of the target vessel proximate the everted end of the graft vessel; and
disconnecting the deployer assembly from the connector assembly;
whereby to hold the everted end of the graft vessel and an area of the target vessel adjacent the graft vessel between the connector assembly tines and the connector assembly seal portion.

19. A method for joining a graft vessel to an opening in a target vessel, the method comprising the steps of:
providing a connector assembly comprising:
a tubular connector member provided with a plurality of tines, the tines in an unstressed state extending inwardly toward a central axis of the connector member, the tines being provided with vessel engaging structure;
a spreader portion comprising a body movable in the connector member to engage the tines and extend the tines in directions outwardly from the connector member central axis; and
a seal portion disposed around the connector member and provided with vessel engaging structure;
providing a deployer assembly for holding, manipulating, and releasing the connector assembly;
attaching the connector assembly to the deployer assembly;
manipulating the deployer assembly to move the spreader portion of the connector assembly to engage the tines and extend the tines outwardly from the connector member central axis;
extending the graft vessel through the deployer assembly and centrally of the outwardly extending tines with a distal end of the graft vessel extending distally of the distal end of the connector assembly;
manipulating the deployer assembly to withdraw the spreader portion of the connection assembly to disengage from the tines to permit the tines to resume the unstressed state;
everting the distal end of the graft vessel back upon the tubular connector member;
producing an opening in the target vessel if one is not already available;
manipulating the deployer assembly to move the everted end of the graft vessel through the target vessel opening, into the target vessel, and into engagement with an interior wall of the target vessel around the opening therein;
manipulating the deployer assembly to move the connector assembly seal portion into engagement with an exterior wall of the target vessel proximate the everted end of the graft vessel; and
disconnecting the deployer assembly from the connector assembly;
whereby to hold the everted end of the graft vessel and an area of the target vessel adjacent the graft vessel between the connector assembly connector member and the connector assembly seal portion.

20. The method in accordance with claim 19 wherein moving the everted end of the graft vessel through the target vessel opening and into the target vessel comprises moving the tines outwardly into penetrating engagement with the graft vessel and moving the connector member and the everted end of the graft vessel affixed to the tines into the target vessel; and
wherein moving the everted end of the graft vessel into engagement with the interior wall of the target vessel comprises moving the tines into penetrating engagement with the target vessel interior wall.
